(19) United States
(12) Patent Application Publication
(10) Pub. No.: US 2015/0176077 A1
(43) Pub. Date: Jun. 25, 2015

(54) METHODS FOR PREDICTING WHETHER A SUBJECT IS AT RISK OF DEVELOPING A FOLLICULAR LYMPHOMA

(71) Applicants: INSERM (INSTITUT NATIONAL DE LA SANTE ET DE LA RECHERCHE MEDICALE), Paris (FR); UNIVERSITE D'AIX MARSEILLE, Marseille Cedex 07 (FR); CENTRE NATIONAL DE LA RECHERCHE SCIENTIFIQUE (CNRS), Paris (FR); IMPERIAL COLLEGE LONDON, London (GB)

(72) Inventors: Kelly Rachel, London (GB); Paolo Vineis, London (GB); Bertrand Nadel, Marseille (FR); Sandrine Roulland, Marseille (FR)

(21) Appl. No.: 14/416,137

(22) PCT Filed: Jul. 29, 2013
(86) PCT No.: PCT/EP2013/065917

§ 371 Date: Jan. 21, 2015

(30) Foreign Application Priority Data
Jul. 27, 2012 (EP) 12305921.4

(51) Int. Cl. C12Q 1/68

(52) U.S. Cl. CPC C12Q 1/6886; C12Q 2600/16; C12Q 2600/156

(57) ABSTRACT

The present invention relates to a method for determining whether a subject is at risk of developing a FL comprising the steps consisting of i) determining the t(14:18) frequency in a blood sample obtained from the subject, ii) comparing the t(14:18) frequency determined at step i) with a predetermined reference value and iii) concluding that the subject has very high probability to develop a FL when the t(14:18) frequency determined at step i) is higher than the predetermined reference value.

### FIELD OF THE INVENTION

The present invention relates to methods for predicting whether a subject is at risk of developing a follicular lymphoma (FL).

### BACKGROUND OF THE INVENTION

FL is the second most common form of non-Hodgkin's lymphoma, accounting for around 20-30% of all cases. FL is characterized histologically by the replacement of normal lymph node architecture with nodular collections of small cleaved and large non-cleaved neoplastic B cells. The t(14;18) chromosomal translocation is seen in 80-90% of FL cases and constitutes the initiating event of a causative cascade leading to FL. This translocation joins the BCL-2 gene with immunoglobulin (Ig) heavy chain locus, resulting in over-expression of the anti-apoptotic BCL-2 protein and extended cell survival. Despite considerable therapeutic efforts developed during the last decades (including combination immunotherapies such as Rituximab-CHOP), it still remains to date an incurable cancer. The clinical course is indeed often indolent and insidious, progressing asymptomatically over many years. Consequently, the diagnosis is frequently delayed, and the treatment performed on a largely disseminated and refractory tumor. It is now clear that prevention and treatment of this cancer is to date largely dependent on the identification of pertinent early biomarkers. t(14;18) translocations are present in blood from healthy individuals, but there is a trend of increased prevalence and/or in farmers exposed to pesticides, a group associated with higher risk of t(14;18)+ non-Hodgkin's lymphoma development. Thus a direct connection between agricultural pesticide use, t(14;18) in blood, and malignant progression was recently investigated (Agopian J, Navarro J M, Gac A C, Lecluse Y, Briand M, Grenot P, Gauduchon P, Ruminy P, Lebailly P, Nadel B, Roulland S. Agricultural pesticide exposure and the molecular connection to lymphomagenesis. J Exp Med. 2009 Jul. 6;206(7):1473-83. Epub 2009 Jun. 8). The data establish that expanded t(14;18)+ clones constitute bona fide precursors at various stages of FL development, and provide a molecular connection between agricultural pesticide exposure, t(14;18) frequency in blood, and clonal progression. However the question “how to identify the subjects bearing the translocation t(14;18) that are at risk of developing FL ?” still remains relevant.

### SUMMARY OF THE INVENTION

The present invention relates to a method for determining whether a subject is at risk of developing a FL comprising the steps consisting of i) determining the t(14:18) frequency in a blood sample obtained from the subject, ii) comparing the t(14:18) frequency determined at step i) with a predetermined reference value and iii) concluding that the subject has very high probability to develop a FL when the t(14:18) frequency determined at step i) is higher than the predetermined reference value.

### DETAILED DESCRIPTION OF THE INVENTION

To identify whether t(14;18) could be used as an early predictor for follicular lymphoma (FL) development, we screened 205 cancer-free subjects enrolled in the prospective EPIC cohort, 62 which developed FL 1-11 years later, and 143 matched controls. We find significant increases in t(14;18) prevalence and frequency among pre-diagnostic FL samples, and define those with a frequency over 1×10−4 to have a 15-fold higher risk of FL development (95%CI=4.5-57). Molecular backtracking in paired pre-diagnostic/tumor samples demonstrates FL progression from the identified t(14;18)+ precursors. This defines the first predictive biomarker for lymphoma.

The present invention relates to a method for determining whether a subject is at risk of developing a FL comprising the steps consisting of i) determining the t(14:18) frequency in a blood sample obtained from the subject, ii) comparing the t(14:18) frequency determined at step i) with a predetermined reference value and iii) concluding that the subject has very high probability to develop a FL when the t(14:18) frequency determined at step i) is higher than the predetermined reference value.

The terms “subject,” and “patient,” used interchangeably herein, refer to a male or female. Typically the subject being at risk of developing a FL is a substantially healthy subject but may exhibit one or more risk factors for FL such as for example age, genetic predispositions (Skibola Nat Gen 2009 August;41(8):873-5; Conde Nat Gen 2010 August;42(8):661-4), or environmental factors such as exposure to pesticides (e.g. farmers).

As used herein, the term “t(14:18)” has its general meaning in the art and refers to the translocation involving the BCL2 proto-oncogene (chromosome 18) and the nonexpressed IgH allele (chromosome 14) as described by Tsujimoto et al. (Y. Tsujimoto, J. Gorham, J. Cossman, E. Jaffe, C. M. Croce The t(14;18) chromosome translocations involved in B-cell neoplasms result from mistakes in VDJ joining. Science, 229 (1985), pp. 1390-1393). Thus, a “t(14:18)+ cell” refers to a (14;18)-bearing cell.

As used herein, the term “t(14:18) frequency” refers to the presence of one t(14;18)-bearing cell (i.e. a t(14:18)+ cell) per the number of peripheral blood cells [e.g. peripheral blood mononuclear cells (PBMCs) or cells comprised in the fraction of an anticoagulated blood sample after density gradient centrifugation (buffy coat)]. For example, a frequency of 10⁻⁶ represents one t(14;18)-bearing cell in a million of peripheral blood cells.

Methods for determining t(14:18) frequency in a blood sample are well known in the art. Briefly, to provide an accurate and reliable estimation of the frequency of t(14;18)-positive cells in a subject, genomic DNA will be extracted from blood samples (buffy coat or PBMCs) and tested for the presence of t(14;18) translocation using a quantitative real-time BCL2/JH PCR assay (Q-PCR). As most BCL2 breakpoints arise in the Major Breakpoint Region (MBR), located in the 3′ untranslated region of BCL2 exon III, Q-PCR will be performed using primers flanking the 5′ BCL2-MBR in combination with the 3′ JH consensus reverse sequence from the IGH locus and a fluorescent probe matching the MBR region, as previously described (Agopian J, Navarro J M, Gac AC, Lecluse Y, Briand M, Grenot P, Gauduchon P, Ruminy P, Lebailly P, Nadel B, Roulland S. Agricultural pesticide exposure and the molecular connection to lymphomagenesis. J Exp Med. 2009 Jul. 6;206(7):1473-83). Standard curves with serial dilutions of known starting copy number (106-100) were established from a cloned t(14;18)-mbr breakpoint sequence (derived from the Karpas-422 cell line) and a cloned Glyceraldehyde-3-phosphate dehydrogenase (GAPDH)-specific sequence used as a housekeeping gene. For each subject, the Q-PCR BCL2/JH screening consisted in triplicate reactions containing 500 ng target DNA and duplicate reactions for the GAPDH gene. In a typical experiment, the t(14;18) frequency per cell (F) is calculated according to F=2*number of BCL2/JH copies/number of GAPDH copies, where the GAPDH copy number determine the absolute number of cells in a given test sample. The Q-PCR approach described here will detect the t(14;18) translocation with a sensitivity of one copy in 1.5 μg of DNA, i.e., one t(14;18)-positive cell in 250,000 normal cells.

In a particular embodiment, the t(14;18) frequency is determined as described in the EXAMPLE 1.

Predetermined reference value used for comparison may consist of a threshold frequency value. The threshold frequency value may be chosen in order to obtain the optimal sensitivity and specificity, i.e. the benefice/risk balance (clinical consequences of false positive and false negative). Typically, the optimal sensitivity and specificity (and so the threshold value) can be determined using a Receiver Operating Characteristic (ROC) curve based on experimental data. In a particular embodiment the predetermined value is higher than or equal to 10⁻⁴. Typically, the predetermined value is 1.10⁻⁴, 2.10⁻⁴, 3.10⁻⁴, 4.10⁻⁴ or 5.10⁻⁴.

Typically a subject having a frequency higher than 1×10⁻⁴ has a 15-fold higher risk of FL development.

In some embodiments, the method of the invention is performed by a laboratory that will generate a test report. The test report will thus indicates the t(14:18) frequency. In some embodiments, the test result will include a probability score. Typically, the method for calculating the score is based on statistical studies performed on various cohorts of patients. The score may also include other various patient parameters (e.g., age, gender, ethnicity, genetic predisposition, or environmental factors such as exposure to pesticides). The weight given to each parameter is based on its contribution relative to the other parameters in explaining the inter-individual variability of having FL in the relevant disease population. In some embodiments, the test report may be thus generated by a computer program for establishing such a score.

The inventors now provide evidences that t(14:18) frequency represent an early biomarker for FL.

For example, the high-risk subjects identified by the method of the present invention could then be integrated into the current clinical management circuit for asymptomatic FL patients, and ultimately could benefit from the novel targeted strategies designed to delay and/or prevent FL development.

Typically once a high-risk subject is identified by the method of the present invention, a full exploration of his body (e.g. neck, thorax, abdomen and pelvis) may be performed for identifying existence of lymph nodes. Such an exploration may be carried out by palpation or throughout imaging techniques such as computed tomography (CT) scan, or positron emission tomography (PET) scan. Routine blood analysis may also be performed such as blood count. If a lymph node is detected a surgical specimen/excisional lymph node biopsy is performed for diagnosis purposes. A core biopsy may also be performed in subjects without accessible lymph nodes. Once the diagnosis of FL is done, the staging is given according to the current clinical classification (e.g. Ann Harbor system) and watchful waiting or lines of treatment may be finally planed.

Watchful waiting may be preferred may be considered as an appropriate approach in subjects with asymptomatic advanced stage FL in an attempt to delay the need for an aggressive line of treatment. This is particularly the case for subject over 70 years of age. Typically, treatment lines comprise radiotherapy, chemotherapy and immunotherapy. Radiotherapy is regarded as the standard of care for patients with newly diagnosed, limited staged disease. Standard regimen of chemotherapy may be used, such as CHOP (cyclophosphamide, doxorubicin, vincristine, prednisone). In some embodiment, the combination may comprise a monoclonal antibody such as rituximab, alemtuzumab, human or humanized anti-CD20 antibodies, lumiliximab, anti-TRAIL, bevacizumab, galiximab, epratuzumab, SGN-40, and anti-CD74 antibodies. Accordingly standard regiment of chemotherapy and biotherapy may be suitable such as R-CHOP (rituximab plus CHOP).

Alternatively, as recently demonstrated B cell depleting therapy for asymptomatic patients diagnosed for early forms of FL may be preferred (Jegalian A G, Eberle F C, Pack S D, Mirvis M, Raffeld M, Pittaluga S, Jaffe E S. FL in situ: clinical implications and comparisons with partial involvement by FL. Blood. 2011 Sep. 15;118(11):2976-84. Epub 2011 Jul. 18; Kirit M et al. An Intergroup Randomised Trial of Rituximab Versus a Watch and Wait Strategy In Patients with Stage II, III, IV, Asymptomatic, Non-Bulky FL (Grades 1, 2 and 3a). A Preliminary Analysis, ASH 2012). In particular, Jegalian A G. et al. show that asymptomatic patients treated with a B cell depleting agent, such as rituximab, never progress to FL (7 years follow-up) contrary to the major part of the asymptomatic patients who were not treated with the B cell depleting agent (53%). The B cell depleting agent is an anti-B cell antibody, preferably a monoclonal antibody (e.g. a chimeric, humanized or human antibody). For example, a suitable anti-B cell antibody can be an antibody targeting any B cell surface marker e.g. an antiCD20 monoclonal antibody [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], an anti-CD79a antibody, an anti-CD27 antibody, or an antiCD19 antibody (e.g. U.S. Pat. No. 7,109,304). Another example of anti-B cell antibody include an antibody targeting a B cell survival factor or a cytokine imperative for B cell function or an effector thereof (e.g., a receptor which binds the aforementioned factor). Such antibodies include the anti-BAFF-R antibody (e.g. Belimumab, GlaxoSmithKline), the anti-APRIL antibody (e.g. anti-human APRIL antibody, ProSci inc.), the anti-IL-6 antibody [previously described by De Benedetti et al., J Immunol (2001) 166: 4334-4340 and by Suzuki et al., Europ J of Immunol (1992) 22 (8) 1989-1993, fully incorporated herein by reference], the anti-IL-7 antibody (R&D Systems, Minneapolis, Minn.) or the SDF-1 antibody (R&D Systems, Minneapolis, Minn.).

In some embodiments, the high-risk subjects identified by the method of the present invention may be directly administered with a B cell depleting agent for prophylactic purposes.

Accordingly, a further aspect of the invention relates to a B cell depleting agent for use in the prophylactic treatment of FL in a subject considered at risk of developing a FL according to the method of the present invention.

The invention will be further illustrated by the following figures and examples. However, these examples and figures should not be interpreted in any way as limiting the scope of the present invention.

### FIGURES

FIG. 1: t(14;18) status in blood from healthy individuals that subsequently developed FL (Hi-FL) or not (controls).

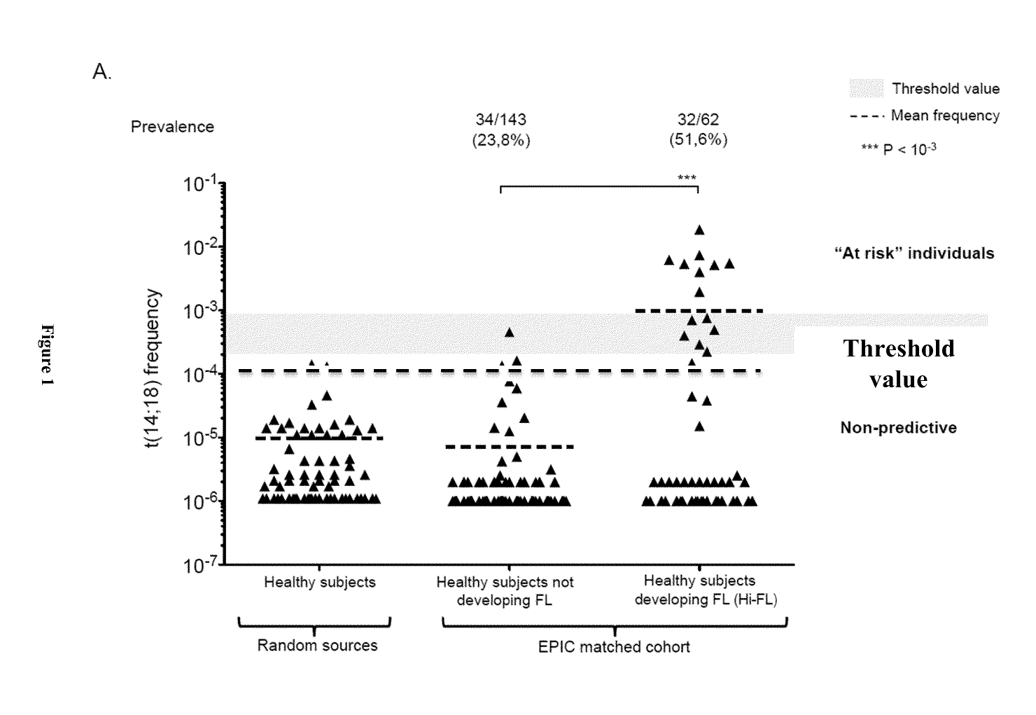

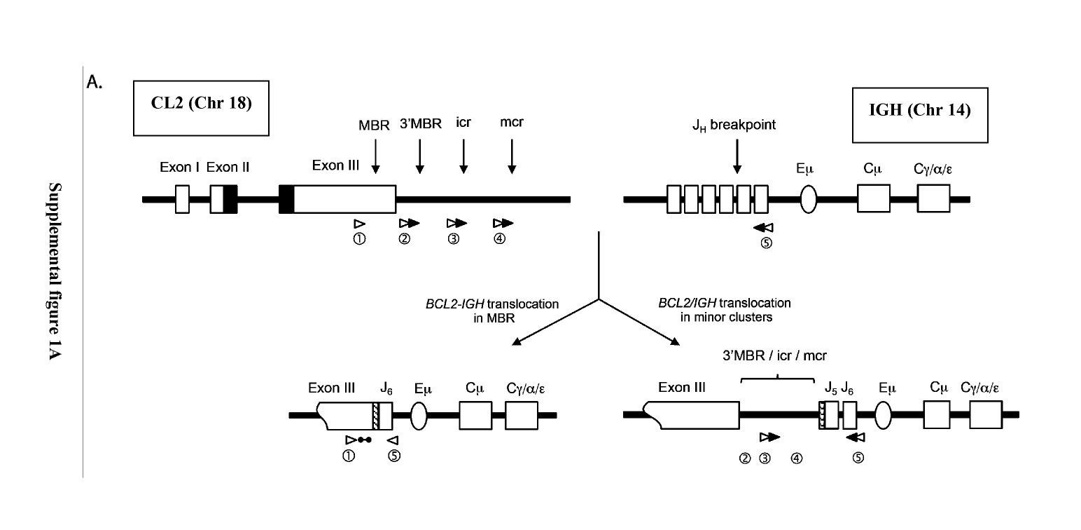

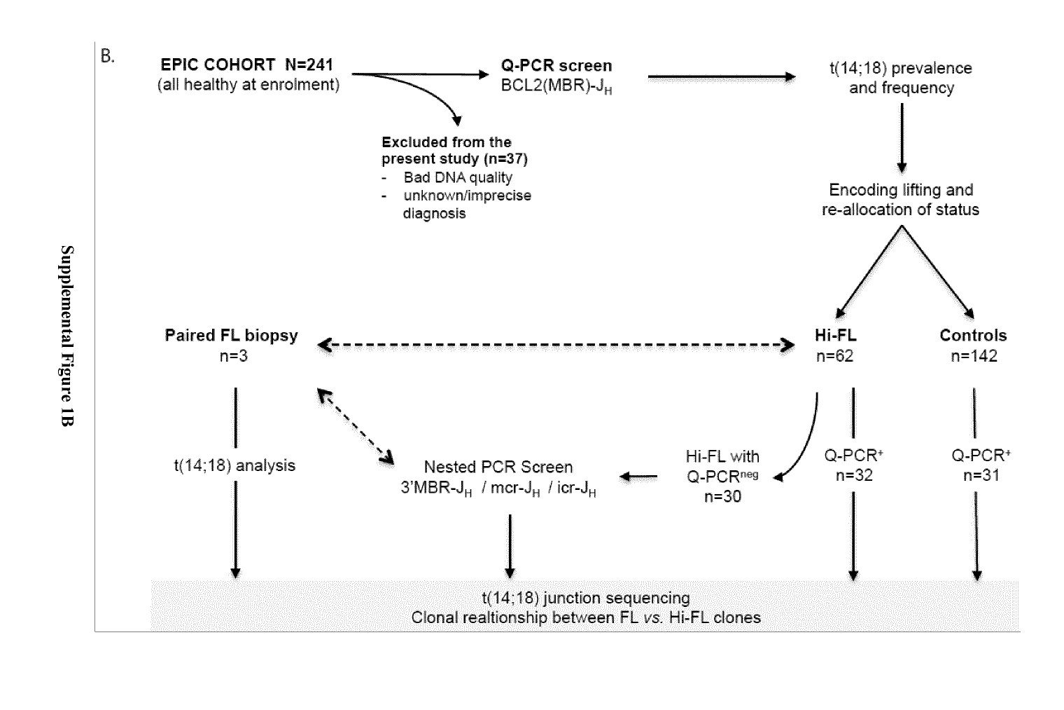

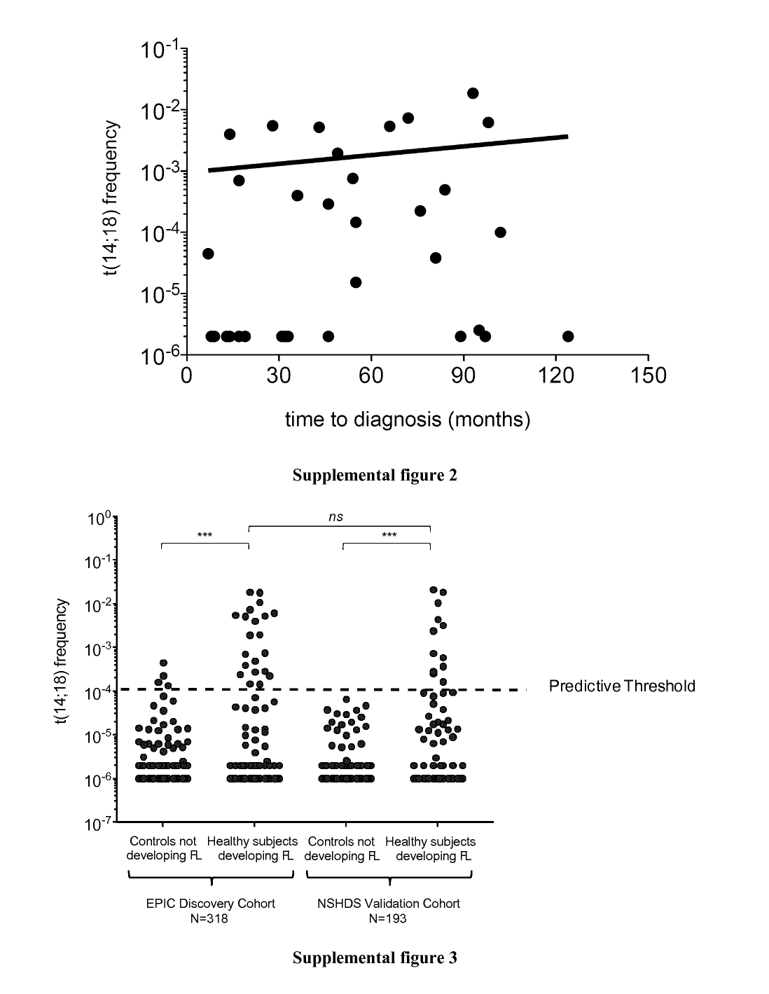

Supplemental FIG. 1 (A) Schematic diagrams of BCL2 and immunoglobulin JH breakpoint regions involved in t(14;18) translocations. The IGH breakpoint is consistently in one of the six joining regions. The BCL2 breakpoints are variable and indicated by vertical arrows: major breakpoint region (MBR), located within the 3′ non-coding region of exon 3; minor cluster region (mcr), located 20-30 kb 3′ to the MBR; and additional clusters 3′ MBR and intermediate cluster region (icr) in between. Bottom left: BCL2/IGH rearrangement at the MBR, bottom right: BCL2/IGH rearrangement at minor breakpoint regions. Positions of MBR \textcircled{0}, 3′MBR \textcircled{2}, icr \textcircled{3} and mcr \textcircled{4} primers for PCR are indicated by horizontal red arrows. Consensus J_{H} primers are indicated by blue arrows. Empty arrow: 1^{st} round PCR, filled arrow: 2^{nd} round internal nested PCR. BCL2-MBR probe used for Q-PCR is depicted in green. Gray shading indicates BCL2 coding exon regions. Dashed boxes at BCL2-IGH junctions indicate N-nucleotide additions. (B) t(14;18) screening workflow in the blood from the EPIC cohort.

Supplemental FIG. 2: Correlation between t(14;18) frequency in prediagnostic samples and time to diagnosis. Only samples with t(14;18)-samples are depicted.

Supplementary FIG. 3: t(14;18) status in blood from healthy individuals that subsequently developed FL or not (controls).The data are given for an expanded discovery EPIC cohort (N=318) and for a validation cohort including 193 new healthy individuals enrolled into the NHSDS cohort. The Northern Sweden Health and Disease Study includes a total of 95,000 healthy individuals aged 40-60 invited for inclusion between 1990 and 2006. Incident cases of FL were identified by linkage with the Swedish Cancer Registry and the same eligibility criteria as for the EPIC cohort was applied to identify suitable cases and their controls. These 68 cases and 125 controls constitute the validation cohort. ***P<10−3 (Mann-Whitney test), ns not significant.

### EXAMPLE 1

### Material and Methods

Study Population:

We performed a case-control study on follicular lymphoma risk, nested within the EPIC cohort. EPIC is a multicenter prospective cohort established to investigate the role of biological, dietary, lifestyle, and environmental factors in the etiology of cancer and other chronic diseases. Approximately 500,000 healthy males and females, aged 35-70 years, were recruited between 1992 and 2000 from 23 centers in 10 European countries, including Denmark, France, Germany, Greece, Italy, the Netherlands, Norway, Spain, Sweden, and the United Kingdom. Details of the EPIC study have been previously published¹¹.

Case Identification for Follicular Lymphoma:

Follow-up of cohort members for these analyses was performed through 2000 to 2004, depending upon the study center. Cases eligible for this study were participants who were recorded with FL, according to the ICOD-3 classification, and had a prediagnostic cryopreserved buffy coat sample. Among men and women in the full EPIC cohort, a total of 81 incident FL cases were observed with available pre-diagnostic samples. 19 participants were excluded because of altered DNA quality (n=4) or discordant ICOD-3 classification code without distinction between de novo DLBCL or DLBCL transformated from FL (n=15). Two controls per case (n=160) were randomly chosen among EPIC cohort who were alive and cancer-free at the time of the cancer diagnosis of the corresponding FL and who matched with age, sex and study center. 17 participants with degraded and non-amplifiable DNA were removed from analysis. The EPIC study and the present FL study was approved by the Ethical Review Board from the International Agency for Research on Cancer and each of the 23 EPIC study centres. Written consent was obtained from all EPIC participants at enrolment into the study.

DNA Isolation:

Genomic DNA was extracted from 241 frozen buffy coats. DNA from FFPE tumor biopsies was isolated using QIAamp DNA FFPE tissue kit (Qiagen). DNA quantity was assessed using a spectrophotometer (Nanodrop). To access the FFPE DNA quality on the 3 tumor biopsies, a gel-based multiplex PCR assay was performed that allows the co-amplification of four PCR fragments ranging from 100 to 400 bp to determine the maximum size of amplifiable DNA fragments. A sample with 4 amplification bands indicates a high quality DNA while a sample with only lower amplification bands or no amplification indicate a low quality DNA.

Quantitative Real-Time PCR for t(14;18)-MBR Translocation:

Quantitative real-time PCR for the t(14;18)-MBR translocation was carried out in buffy coat samples as described previously using the ABI PRISM 7500 Sequence Detection System (PE Applied Biosystems). PCR of the GAPDH reference gene was carried out as a test for amplifiable DNA and to determine the number of cells analyzed in each single assay. Both primers and probes were previously reported⁵. The PCR mixture contained each forward and reverse primer at a concentration of 300 nM, probe at a concentration of 200 nM, the standard TaqMan Universal PCR Master Mix and 500 ng DNA in each PCR replicates. After a 2-min incubation at 50° C. to allow for cleavage by Uracil-N-Glycosylase (UNG) and incubation at 95° C. for 10 min, each PCR cycle consisted of 15 sec denaturation at 95° C., and 1 min of combined annealing/extension at 60° C. for 50 cycles. Standard curves were established for the t(14;18)-MBR translocation-specific PCR, as well as for the GAPDH-specific PCR as described. For each buffy coat samples, the screening consisted of 3 PCR reactions with 500 ng DNA for t(14;18), 2 PCR replicates with 100 ng for the reference GAPDH gene, 2 positive t(14;18)⁺ cell lines (RL7 and Karpas 422) and 1 negative control (PCR mixture alone). The quantitative real-time PCR technique described here can detect the t(14;18)-translocation with a sensitivity of one copy in 1.5 μg of DNA, i.e., one t(14;18)-positive cell in 250,000 normal cells.

Quantitative Fluctuation-PCR for Alternative t(14;18) Breakpoints:

A sensitive nested PCR assay was performed in Q-PCR^{neg} Hi-FL to determine the presence of alternative BCL2-IGH rearrangements within the icr, mcr and 3′MBR breakpoint clusters where heterogeneous amplicon PCR lengths (>250 bp) are not compatible with real-time PCR assay (Supplementary FIG. 1A).

Clonal Validation by DNA Sequencing:

BCL2-IGH junctions amplified by PCR either from controls, prediagnostic FL and FL tumor samples were cloned and subjected to conventional Sanger-based sequencing of PCR products.

Statistical Analyses:

Fisher test for prevalence

Mann-Whitney test to compare frequency between Hi-FL and controls.

### Results and Discussion

FL, the second most common adult B-cell lymphoma in westernized countries, is usually characterized by an indolent clinical course, evolving asymptomatically over many years. Consequently, the diagnosis is frequently delayed, and the treatment performed on a largely disseminated tumor. Although the advent of highly effective therapies and the availability of anti-CD20 antibodies, such as Rituximab, has significantly improved clinical outcome, FL remains incurable and patients continue to die from the disease following resistance to treatments or transformation into a more aggressive diffuse large B-cell lymphoma¹. FL represent a particular attractive model to study early phases of cancer development because the acquisition of the genetic hallmark t(14;18) translocation—the earliest recurrent event of FL genesis giving rise to a BCL2/IGH fusion—originate from a bone marrow pre-B cell while the malignant FL clones derive from the transformation of germinal center (GC) B-cells in secondary lymphoid organs². Accordingly, a large proportion (>70%) of healthy individuals harbor low levels of circulating 414;18)⁺ cells, yet will never develop FL, indicating that BCL2 ectopic expression is necessary but not sufficient for tumor progression³. The relationship between t(14;18) and progression to disease however remains to date unclear. In rare cases, “healthy” individuals carry unusually high frequencies of t(14;18) (10 to 100 times more than the average population)⁴, especially when exposed to environmental risk factors for lymphoma⁵, and sporadic cases of FL progression have been reported⁶⁻⁸. We previously demonstrated that such t(14;18)^{high} cells constitute an expanding clonal population of atypical B-cells, issued from the GC, and sharing illegitimate genotypic and phenotypic features exclusively seen in FL^{5,9}. Nevertheless, a key gap in our understanding is whether t(14;18)⁺ cells with “FL-like” features in healthy individuals constitute clonally-related FL precursors, and if high t(14;18) frequencies in blood represent a suitable biomarker of FL progression.

Considering that FL is diagnosed in ˜1/25,000 individuals annually, and that FL likely develops over several decades, prospective cancer cohort studies provide a unique opportunity to detect and quantify t(14;18) before diagnosis and to investigate the association of a predictive molecular biomarker with disease outcome¹⁰. We thus took advantage of the European Prospective Investigation into Cancer and Nutrition cohort (EPIC) that included more than 520,000 healthy participants enrolled between 1991 and 2000, with an active follow-up (>15 years) for cancer incidence¹¹. Among EPIC participants, we identified 62 subjects that developed FL during follow-up (Hi-FL) and had good-quality archived prediagnostic blood sample collected 2 months to 10.4 years before diagnosis, and selected a matched control (cancer-free) population of 143 subjects (>2 controls/case). A double-blinded screening PCR-based assay was used to quantify t(14;18)⁺ frequencies in encoded prediagnostic samples from the 205 healthy participants (see methods). The assay enabled high sensitivity (<1 translocation/500,000 cells) and covered ˜70% of BCL2 breakpoints (Supplementary FIG. 1). Upon lift of encoding, samples were reallocated among control and Hi-FL groups, and revealed significantly higher t(14;18) prevalence and frequencies in Hi-FL compared to controls (FIG. 1). Similar highly significant results were obtained when t(14;18)-negative samples were removed from the analysis. Although t(14;18) frequencies varied over a 4-log range (from undetectable to one every 100 cells), the higher frequency quartile in Hi-FL reached levels unseen in the control group and never previously observed in blood from large screenings of healthy subjects⁴. Based on this comparative analysis, a conservative high-risk predictive subgroup for FL development could be defined above a frequency threshold of 5.10⁻⁵, encompassing ˜18% of the Hi-FL group (and none of the controls).

We define those with a frequency over 1×10⁻⁴ to have a 15-fold higher risk of FL development (95%CI=4.5-57).

Importantly, a proportion of subjects with lower and/or undetectable t(14;18) also developed FL during follow-up, suggesting that not all patients display a (continuing) rise of circulating t(14;18)-cells during FL progression. To further delineate this possibility, we first refined our screen. Although 85% of FLs harbor translocations in the major breakpoint region (BCL2(MBR)/J_{H}), about 10-15% use alternative BCL2 breakpoints or lack t(14;18) altogether. We thus designed a sensitive nested PCR assay to screen for three additional breakpoint clusters (mcr, 3′MBR and icr, Supplementary FIG. 1A), each involved in <10% of FL cases. Four new cases were identified among 30 screened Hi-FL samples (two mcr/JH and two 3′MBR/JH).

We next asked whether increased t(14;18) frequency might be preferentially observed in pre-diagnostic samples harvested close to diagnosis. Elapsed time between inclusion and diagnosis in the Hi-FL group ranged from<2 months to>10 years (average 4,6 years), and the median time to diagnosis was not different according to t(14;18) status (P=0.3). No significant correlation was either observed when selecting only the t(14;18)⁺ cases (Supplementary FIG. 2, P=0.18, r²=0.05). Furthermore, no significant age-effect was observed (not shown).

We next sought to determine whether the t(14;18)⁺ clones present in the prediagnostic samples constituted the early precursors which had been progressing to overt FL. Three FL biopsies from EPIC subjects were obtained, their t(14;18) clonotypic breakpoints determined, and compared to the ones found in the corresponding prediagnostic samples. All FL biopsies were found t(14;18)-positive, included one mcr variant, and corresponded to t(14;18)⁺ prediagnostic samples with low, high or very high frequencies. The same t(14;18) clonotype was systematically found before and after FL outcome, demonstrating that “FL-like” cells in blood from healthy donors constitute bona-fide FL precursors, some of which will progress to FL. Among healthy individuals bearing such precursor clones, we evaluate that those displaying t(14;18) levels reaching one every 2000 blood cells might be considered at high risk for FL progression in a 1-15 years' time-frame. Further investigation is needed to evaluate the evolution over time of t(14;18) levels at the individual level, to further characterize candidate oncogenic abnormalities associated with asymptomatic precursor conditions and relevant to FL progression^{12,13}, and to identify additional biomarkers allowing to detect healthy individuals in which FL progression is devoid of t(14;18) rise in blood. Overall, the early detection of “high-risk” individuals opens great clinical opportunities for active surveillance and/or intervention in a critical window, prior to the occurrence of manifest FL. Throughout increasingly refined imaging technologies, such high-risk individuals might profit from integrating the current clinical management circuit for asymptomatic FL patients, and ultimately benefiting from novel targeted strategies designed to delay and/or prevent FL development.

SUPPLEMENTARY TABLE 1
Summary of the Follicular Lymphoma-EPIC study populations.

| | EPIC COHORT | |
|---|---|---|
| | | Cases who |
| | Controls | developed FL |
| N | 143 | 62 |
| Age at blood draw, median | 55.9 (38.5-77.5) | 54.2 (39-77) |
| (range), y | | |
| Age at diagnosis, median | — | 57.5 (41-79) |
| (range) | | |
| Sex | 49M/94F | 23M/39F |
| Calendar years of sampling | 1993-2002 | 1993-2001 |
| Calendar years of diagnosis | — | 1997-2004 |
| Time from blood collection to | — | 50.5 (2-124) |
| diagnosis, median (range), mo | | |
| t(14; 18) characteristics | | |
| Prevalence | 34/143 (23.8%) | 32/62 (51.6%) |
| Mean frequency (×10⁻⁵) | 0.8 | 97 |
| Mean frequency in positive | 3 | 180 |
| samples | | |
| Range (×10⁻⁵) | 0.1-45 | 0.1-1900 |

^{a}Controls were individually matched on age, and recruitment centre. Five individuals from the group who developed FL and 17 individuals from the control group were excluded from analyses because of DNA quality unsuitable for PCR amplification. 15 individuals encoded as samples who developed DLBCL w/o further information were also removed from the cases group.

### EXAMPLE 2

We have now incremented the size of the cohort to 511 healthy individuals issued from the EPIC cohort and the NSHDS cohort, including 165 who developed FL later on (2-241 months) and 346 controls (Supplementary FIG. 3). The new ROC curve analysis confirmed the 1×10⁻⁴ threshold as predictive for FL development. The risk estimate for the pooled analyses was 15.43 (95% CI 7.17, 33.21, p=2.59×10−12; total sample of 165 cases and 446 controls).

### REFERENCES

Throughout this application, various references describe the state of the art to which this invention pertains. The disclosures of these references are hereby incorporated by reference into the present disclosure.
1. Relander, T. et al. Prognostic factors in follicular lymphoma. J. Clin. Oncol. 28, 2902-13 (2010).
2. Shaffer Iii, A. L., Young, R. M. & Staudt, L. M. Pathogenesis of Human B Cell Lymphomas. Annual review of immunology (2011).
3. Roulland, S. et al. Early steps of follicular lymphoma pathogenesis. Advances in immunology 111, 1-46 (2011).
4. Schuler, F. et al. Prevalence and frequency of circulating t(14;18)-MBR translocation carrying cells in healthy individuals. Int J Cancer 124, 958-63 (2009).
5. Agopian, J. et al. Agricultural pesticide exposure and the molecular connection to lymphomagenesis. J Exp Med 7, 1473-83 (2009).
6. Bretherick, K. L. et al. Elevated circulating t(14;18) translocation levels prior to diagnosis of follicular lymphoma. Blood 116, 6146-7 (2010).
7. Weigert, O. et al. Molecular Ontogeny of Donor-Derived Follicular Lymphomas Occurring after Hematopoietic Cell Transplantation. Cancer Discovery (2011).
8. Hart, J. et al. Transmission of a follicular lymphoma by allogeneic bone marrow transplantation—evidence to support the existence of lymphoma progenitor cells. British journal of haematology 136, 166-7 (2007).
9. Roulland, S. et al. Follicular lymphoma-like B cells in healthy individuals: a novel intermediate step in early lymphomagenesis. The Journal of Experimental Medicine 203, 2425-2431 (2006).
10. Landgren, O. et al. B-cell clones as early markers for chronic lymphocytic leukemia. N. Engl. J. Med. 360, 659-667 (2009).
11. Riboli, E. et al. European Prospective Investigation into Cancer and Nutrition (EPIC): study populations and data collection. Public Health Nutr 5, 1113-24 (2002).
12. Morin, R. D. et al. Frequent mutation of histone-modifying genes in non-Hodgkin lymphoma. Nature 476, 298-303 (2011).
13. Oricchio, E. et al. The Eph-receptor A7 is a soluble tumor suppressor for follicular lymphoma. Cell 147, 554-64 (2011).

1. A method for determining whether a subject is at risk of developing a follicular lymphoma (FL), comprising the steps consisting of
i) determining a t(14:18) frequency in a blood sample obtained from the subject, and
ii) comparing the t(14:18) frequency determined at step i) with a predetermined reference value,
and iii) concluding that the subject has very high probability to develop a FL when the t(14:18) frequency determined at step i) is equal to or higher than a predetermined reference value of 1×10⁻⁴, 2×10⁻⁴, 3×10⁻⁴, 4×10⁻⁴ or 5×10⁻⁴.

2. The method according to claim 1 wherein said concluding step includes the step of determining that the subject has a 15-fold higher risk of developing a FL relative to a subject with a t(14:18) frequency lower than 1×10⁻⁴.

3. The method according to claim 1, wherein said predetermined reference value is 1×10⁻⁴.

4. A method for determining whether a subject is in need of a prophylactic treatment to inhibit development of a follicular lymphoma (FL), comprising the steps of
obtaining a blood sample from said subject, and
isolating genomic DNA from leukocytes in said blood sample, and
testing quantitatively said genomic DNA for t(14:18) translocations of BCL2/JH to determine a quantity of t(14:18) translocations per number of said leukocytes, and
identifying said subject as being in need of a prophylactic treatment to inhibit development of a FL when said quantity of t(14:18) translocations exceeds 1×10⁻⁴.

5. A method for treating a subject at risk for development of a follicular lymphoma (FL), comprising the steps of
obtaining a blood sample from said subject, and
isolating genomic DNA from leukocytes in said blood sample, and
testing quantitatively said genomic DNA for t(14:18) translocations of BCL2/JH to determine a quantity of t(14:18) translocations per number of said leukocytes, and
identifying said subject as being in need of treatment to inhibit development of a FL when said quantity of t(14:18) translocations exceeds 1×10⁻⁴, and
treating said subject with at least one treatment selected from the group consisting of B-cell depletion, radiation therapy and chemotherapy.
